(19) United States
(12) Patent Application Publication
(10) Pub. No.: US 2015/0018683 A1
(43) Pub. Date: Jan. 15, 2015

(54) ULTRASONIC PROBE HAVING AUXILIARY MONITOR MOUNTED THEREON, AND ULTRASONIC DIAGNOSTIC DEVICE COMPRISING SAME

(71) Applicants: Dong Rak Kwon, Daegu (KR); Gi Young Park, Daegu (KR)

(72) Inventors: Dong Rak Kwon, Daegu (KR); Gi Young Park, Daegu (KR)

(21) Appl. No.: 14/379,601

(22) PCT Filed: Feb. 14, 2013
(86) PCT No.: PCT/KR2013/001152

§ 371 Date: Aug. 19, 2014

(30) Foreign Application Priority Data
Mar. 8, 2012 (KR) 10-2012-0024132

(51) Int. Cl. A61B 8/00; A61B 8/08

(52) U.S. Cl. CPC A61B 8/461; A61B 8/4455; A61B 8/4444; A61B 8/0841; A61B 8/0891
USPC 600/443

(57) ABSTRACT

Disclosed are an ultrasonic probe having an auxiliary monitor mounted thereon and an ultrasonic diagnostic device comprising the same. According to the present invention, the ultrasonic diagnostic device includes: an ultrasonic probe for scanning ultrasonic waves, converting the returned ultrasonic waves into an electric signal, and outputting, on an auxiliary monitor mounted on one side thereof, a part of or the entire image corresponding to the converted electric signal; a main body of the ultrasonic diagnostic device which is provided with the converted electric signal; and a display device which is provided with the image corresponding to the electrical signal from the main body of an ultrasonic diagnostic device so as to output the entirety of the provided image, wherein the ultrasonic probe comprises an injection device for sucking from or injecting blood or an injection liquid into a predetermined target point within the image outputted on the auxiliary monitor.

### TECHNICAL FIELD

The present invention relates to an ultrasonic probe and an ultrasonic diagnostic device including the same, and more particularly, to an ultrasonic probe that has an auxiliary monitor mounted on one side thereof and simultaneously outputs a part or the entirety of an image output on a display device of an ultrasonic diagnostic device through the auxiliary monitor, and has a syringe mounted on one side of the ultrasonic probe and automatically inserts a needle of the syringe into a predetermined blood vessel or target point by using a distance estimated by an ultrasonic diagnostic result, and an ultrasonic diagnostic device including the same.

### BACKGROUND ART

An ultrasonic imaging device is an imaging device that shows the inside of a human body by using ultrasonic waves instead of radioactive rays which are harmless to the human body and can be generally used for a diagnosis and a medical procedure.

In recent years, a portable ultrasonic device having the size of a cellular phone has been developed by decreasing the size of the existing ultrasonic device, and as a result, it will be considered that a usage area thereof will be extended.

Medical procedures using ultrasonic waves include numerous fields including insertion of a catheter into a jugular vein or a subclavian vein, removal of an exudation from or injection of a treatment drug into a joint, a mammotome procedure, removal of the exudation from a pleural cavity or a pericardium, and the like.

However, in all ultrasonic diagnostic devices used in the related art, an image is displayed on a monitor in a device separated from an ultrasonic probe as presented in FIG. 1. That is, a signal acquired by the ultrasonic probe is displayed on a monitor at a different position regardless of where the medical procedure is being actually performed, and as a result, it is difficult to perform the medical procedure when an operator must view a monitor and not a position of his/her hands.

### DISCLOSURE

### Technical Problem

The present invention has been made in an effort to solve the problems in the related art, and an object of the present invention is to provide an ultrasonic probe that has an auxiliary monitor mounted on one side thereof and simultaneously outputs a part or the entirety of an image output on a display device of an ultrasonic diagnostic device through the auxiliary monitor, and an ultrasonic diagnostic device including the same.

Another object of the present invention is to provide an ultrasonic probe that has a syringe mounted on one side thereof and automatically inserts a needle of the syringe into a predetermined blood vessel or at a target point by using a distance estimated by an ultrasonic diagnosis result, and an ultrasonic diagnostic device including the same.

However, the objects of the present invention are not limited to the aforementioned objects, and other objects, which are not mentioned above, will be apparent to those skilled in the art from the following description.

### Technical Solution

In order to accomplish the aforementioned objects, an ultrasonic diagnostic device according to an aspect of the present invention includes: an ultrasonic probe which scans ultrasonic waves, converts the returned ultrasonic waves into an electric signal, and outputs, on an auxiliary monitor mounted on one side thereof, a part of or the entire image corresponding to the converted electric signal; a main body of the ultrasonic diagnostic device which is provided with the converted electric signal; and a display device which is provided with the image corresponding to the electrical signal from the main body of the ultrasonic diagnostic device, and outputs the entirety of the provided image, in which the ultrasonic probe includes an injection device which sucks from or injects blood or an injection liquid at a predetermined target point within the image output on the auxiliary monitor.

The ultrasonic probe may output a central area having a predetermined size in the image output through the display device on the auxiliary monitor.

The ultrasonic probe may control the injection device to estimate distance information up to the predetermined target point in the image output on the auxiliary monitor and suck or inject the blood or the injection liquid into the target point by using the estimated distance information.

An ultrasonic probe having an auxiliary monitor mounted thereon according to another aspect of the present invention includes: a probe head which scans ultrasonic waves, and converts the returned ultrasonic waves into an electric signal; a cable which transfers the converted electric signal to an ultrasonic diagnostic device body, and receives an image corresponding to the electric signal transferred from the ultrasonic diagnostic device body; and an injection device which inserts a syringe into a predetermined target point in an image corresponding to the electric signal and sucks or injects blood or an injection liquid through the inserted syringe.

The auxiliary monitor may display a central area having a predetermined size in an image output through a display device mounted at one side of the probe head and connected to the ultrasonic diagnostic device body.

The auxiliary monitor may be physically coupled with the probe head or physically separated from the probe head to be removably coupled thereto.

An ultrasonic probe having an auxiliary monitor mounted thereon according to yet another aspect of the present invention, includes: a probe head which scans ultrasonic waves, and converts the returned ultrasonic waves into an electric signal; an auxiliary monitor which outputs a part or the entirety of an image corresponding to the converted electric signal; a cable which transfers the converted electric signal to an ultrasonic diagnostic device body and receives an image corresponding to the electric signal transferred from the ultrasonic diagnostic device body; and an injection device which inserts a syringe into a predetermined target point in the image output to the auxiliary monitor, and sucks or injects blood or an injection liquid through the inserted syringe.

The auxiliary monitor may output a central area having a predetermined size in an image output through a display device mounted at one side of the probe head and connected to the ultrasonic diagnostic device body.

The injection device may include fixing means which fixes the syringe for sucking or injecting the blood or the injection liquid.

The injection device may include: fixing means which fixes the syringe for sucking or injecting the blood or the injection liquid; rotating means which rotates the syringe so as to control an angle at which a needle of the syringe is inserted; and driving means which advances or retreats a piston of the syringe inserted at the target point.

The driving means may estimate distance information up to the target point from the ultrasonic head by using the ultrasonic wave and control an advance or retreat degree of the piston of the syringe, which is to be inserted, by using the estimated distance information.

The ultrasonic probe may further include control means which estimates distance information from the probe head to the target point by using the ultrasonic wave, and the injection device may include driving means which controls the advance or retreat degree of the piston of the syringe, which is to be inserted at the target point, by using the distance information estimated by the control means.

### Advantageous Effects

Therefore, according to the present invention, an auxiliary monitor is mounted on one side of an ultrasonic probe and a part or the entire image output on a display device of an ultrasonic diagnostic device is simultaneously output through the auxiliary monitor, and as a result, an operation position and eyes of an operator coincide with each other so as for the operator to perform a convenient and accurate procedure.

Further, according to the present invention, a syringe is mounted on one side of the ultrasonic probe and a needle of the syringe is automatically inserted into a predetermined blood vessel or at a target point by using a distance estimated by an ultrasonic diagnosis result, thereby improving convenience of the operator.

### DESCRIPTION OF DRAWINGS

FIG. 1 is a schematic view of an ultrasonic examination generally performed in the related art.

FIG. 2 is a first diagram illustrating a configuration of an ultrasonic probe according to an embodiment of the present invention.

FIG. 3 is a second diagram illustrating a configuration of an ultrasonic probe according to an embodiment of the present invention.

FIG. 4 is a diagram illustrating a configuration of an injection device according to an embodiment of the present invention.

FIG. 5 is a diagram illustrating a configuration of an ultrasonic diagnostic device according to an embodiment of the present invention.

### BEST MODE

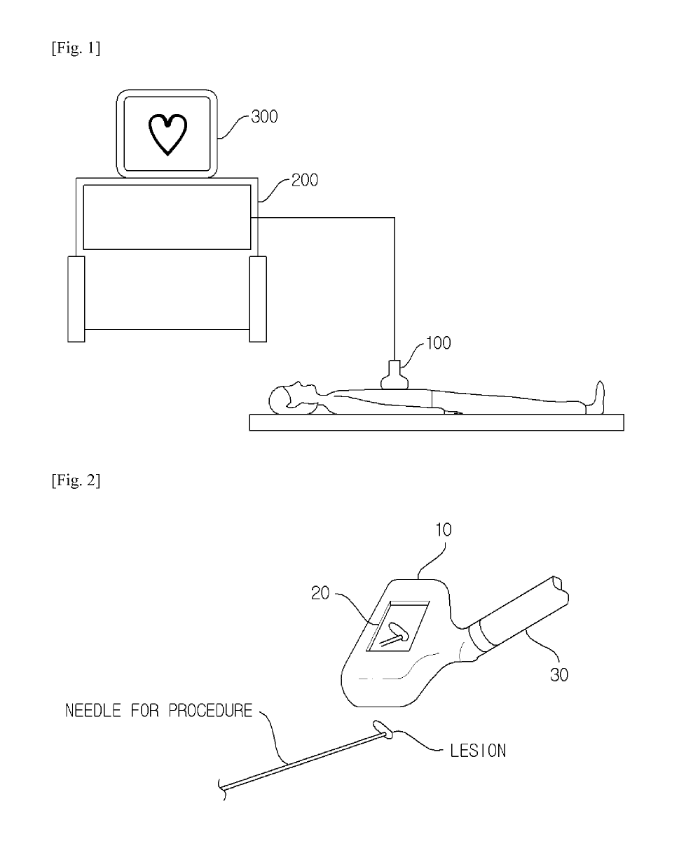

Hereinafter, an ultrasonic probe having an auxiliary monitor mounted thereon and an ultrasonic diagnostic device including the same according to embodiments of the present invention will be described with reference to the accompanying FIGS. 1 to 5. Parts required to understand an operation and an action according to the present invention will be primarily described in detail. Like reference numerals presented in respective drawings refer to like elements throughout the specification. Further, in describing the present invention, well-known related functions or configurations will not be described in detail when it is judged that the detailed description for the well-known related functions or configurations may unnecessarily obscure the understanding of the present invention.

The present invention suggests a new scheme in which (1) an auxiliary monitor is mounted on one side thereof, and a part and the entirety of an image output on a display device of an ultrasonic diagnostic device is simultaneously output through the auxiliary monitor, and (2) a syringe is mounted on one side of the ultrasonic probe and a needle of the syringe is inserted into a predetermined blood vessel or target point by using a distance estimated by an ultrasonic diagnostic result to automatically extract blood.

First, an ultrasonic diagnostic device according to the present invention may be configured to include an ultrasonic probe 100, an ultrasonic diagnostic device body 200, and a display device 300.

The ultrasonic probe 100 may scan ultrasonic waves, convert information loaded on the returned ultrasonic waves into an electric signal, and transmit the converted electric signal to the ultrasonic diagnostic device body 200. In particular, the ultrasonic probe 100 according to the present invention includes an auxiliary monitor for outputting the electric signal, and may further include an injection device for automatically inserting a needle into a blood vessel, an organization, or a target point under skin and fatty tissue.

Further, the ultrasonic probe 100 may estimate distance information up to the blood vessel, the organization, or the target point positioned under the skin and the fatty tissue according to an ultrasonic diagnosis result and control the injection device by using the estimated distance information.

The ultrasonic diagnostic device body 200 may receive the electric signal transmitted from the ultrasonic probe 100 to generate an image signal or image data by using the received electric signal and output the generated image signal through the display device 300. Further, the ultrasonic diagnostic device body 200 may transfer an image signal to the auxiliary monitor.

FIG. 2 is a first diagram illustrating a configuration of an ultrasonic probe according to an embodiment of the present invention.

As illustrated in FIG. 2, the ultrasonic probe 100 according to the present invention includes an ultrasonic probe head 10 that transmits or receives ultrasonic waves, an auxiliary monitor 20, and a cable 30, and the cable 30 is connected to the main body 200 of an ultrasonic diagnostic device.

As the embodiment of the present invention, in the ultrasonic probe 100 constituting the ultrasonic diagnostic device for a medical procedure, the auxiliary monitor 20 having a predetermined size may be mounted on the top of the probe head 10.

In this case, the auxiliary monitor 20 may be configured to be physically coupled with the probe head 10, but the present invention is not limited thereto, and the auxiliary monitor 20 is physically separated from the probe head 10 to be removably coupled thereto, however, may be configured to transmit an image by wired or wireless communication.

The auxiliary monitor 20 may output the entirety or a part of an image output from the ultrasonic diagnostic device body 200 to be described below simultaneously with the display device 300. Accordingly, an operator may perform a procedure while examining the auxiliary monitor 20 put on the probe head 10, thereby enabling a convenient and accurate procedure.

The ultrasonic probe 100 according to the embodiment of the present invention may include, as general components mounted on the existing ultrasonic probe, a piezo-electric resonator, an acoustic matching layer, an acoustic lens, a backing material, and a flexible printing circuit (FPC) therein.

The components will be simply described below. The piezo-electric resonator is elementized into a pamphlet type piezoelectric element by dicing a plate type piezo-electric resonance material.

The acoustic matching layer for matching acoustic impedance is provided at the side of an earth electrode of the piezo-electric resonator, and the acoustic lens is provided on the surface of the acoustic matching layer.

Further, the backing material made of rubber having high sound absorptivity is attached to the side of a signal electrode of the piezo-electric resonator via an epoxy based resin.

The FPCs are disposed on both surfaces of the piezo-electric resonator to face each other. A front end of each FPC is connected to the signal electrode and the earth electrode of the piezo-electric resonator through a solder material.

The FPC is bent around a joint with the piezo-electric resonator at approximately 90 degrees, and a stylobate thereof is connected to the ultrasonic diagnostic device body 200 disposed at the backing material side.

When the ultrasonic probe having the aforementioned configuration is used, the acoustic lens first contacts a diagnosis target and the electric signal is applied to the piezo-electric resonator through the FPC to generate ultrasonic waves from the piezo-electric resonator.

The generated ultrasonic waves are transmitted to the diagnosis target through the acoustic lens, reflected on the inside of the diagnosis target, and then received by the piezo-electric resonator. The received ultrasonic waves are converted into the electric signal in the piezo-electric resonator to be transferred to the ultrasonic diagnostic device body 200 through the cable 30 via the FPC.

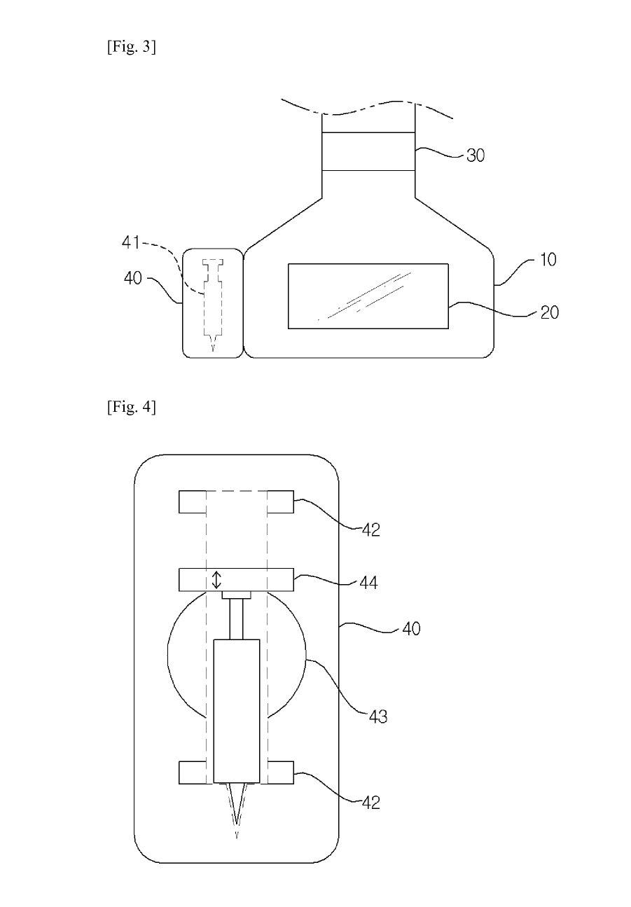

FIG. 3 is a second diagram illustrating a configuration of an ultrasonic probe according to an embodiment of the present invention.

As illustrated in FIG. 3, the ultrasonic probe 100 according to the present invention may be configured to include an ultrasonic probe head 10 that transmits or receives ultrasonic waves, an auxiliary monitor 20, and a cable 30, and an injection device 40.

Since the configuration of the ultrasonic probe 100 according to the present invention has the same content described in FIG. 2 except for the injection device 40, the description of the constitution will be skipped, and only the injection device 40 will be described.

The injection device 40 may be configured to be mounted with a syringe 41 and automatically insert a needle of the syringe 41 into the blood vessel, the organization, or the target pint positioned under the skin and the fatty tissue shown in the image output on the auxiliary monitor 30 or the display device.

In this case, the ultrasonic probe may further include control means (not illustrated). That is, the control means may estimate distance information up to the target point from the probe head by using the ultrasonic waves and transmit the electric signal to the injection device 40 by using the estimated distance information. Then, the injection device 40 may control an advance or retreat degree of a piston of the syringe 41 according to the distance information.

Further, the control means may transmit the electric signal to the ultrasonic diagnostic device body and receive an image signal corresponding to the electric signal to transmit the received image signal to the auxiliary monitor 30

FIG. 4 is a diagram illustrating a configuration of an injection device according to an embodiment of the present invention.

As illustrated in FIG. 4, the injection device 40 according to the present invention may be configured to include fixing means 42 that fixes the syringe, rotating means 43, and driving means 44.

The fixing means 42 as means for fixing the syringe may include, for example, first fixing means for fixing an upper part of the syringe and second fixing means for fixing a lower part of the syringe.

The rotating means 43 may rotate the syringe to control an angle at which the needle of the syringe is inserted. For example, the needle of the syringe may be vertically inserted or obliquely inserted into skin and a fatty tissue at a predetermined angle by controlling the angle.

The reason for controlling the angle at which the syringe needle is inserted is to prevent the blood vessel from being ruptured when the needle of the syringe is vertically inserted into the blood vessel.

The driving means 43 may suck blood or an injection liquid or inject the injection liquid into the blood vessel by inserting the needle of the syringe into the organization by advancing or retreating the piston of the syringe.

In this case, the driving means 43 receives the distance information from the control means of the ultrasonic probe to control the advance or retreat degree of the piston according to the received distance information.

Further, the driving means 43 directly estimates the distance information up to the blood vessel, the organization, or the target point under the skin and the fatty tissue by using the ultrasonic waves to control the advance or retreat degree of the piston by using the estimated distance information.

In this case, in the present invention, a separate driving button is further provided in at one side of the injection device 40 or the ultrasonic probe 100 to drive the syringe as a user presses the separate driving button.

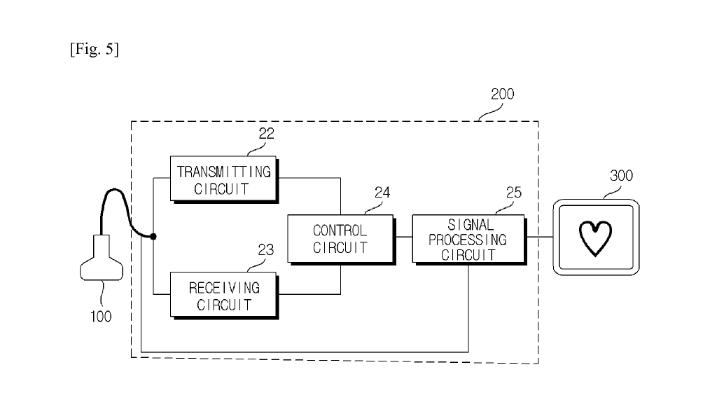

FIG. 5 is a diagram illustrating a configuration of an ultrasonic diagnostic device according to an embodiment of the present invention.

As illustrated in FIG. 5, the ultrasonic diagnostic device 200 according to the present invention may be configured to include a transmitting circuit 22, a receiving circuit 23, a control circuit 24, and a signal processing circuit 25.

The transmitting circuit 22 generates a driving signal for generating the ultrasonic waves and provides the generated driving signal to the piezo-electric resonator in the ultrasonic probe to generate the ultrasonic waves in the resonator.

The receiving circuit 23 delay-adds an echo signal from the piezo-electric resonator.

The signal processing circuit 25 receives the echo signal from the receiving circuit 23 to generate data of a B mode image, data of a D mode (Doppler) image, or data of an M (motion) mode image.

In this case, the ultrasonic diagnostic device may provide a B (brightness) mode image that shows a reflection coefficient of an ultrasonic wave reflected from a target body as a 2D image, a D (Doppler) mode image that show a speed of a moving target body as a Doppler spectrum by using a Doppler effect, a C (color) mode image that shows the speed of the target body that moves as a color by using the Doppler effect, an M (motion) mode image that shows how bio-information (for example, luminance information) of a target body at a specific portion is changed with time in the B mode image, and an elastic mode image that shows a difference in reaction when a compression is applied to the target body and when the compression is not applied, and the like.

The display device 300 displays the generated data of the B mode image, data of the D (Doppler) mode image, or the M (motion) mode image.

In this case, the image output from the signal processing circuit 25 is simultaneously sent to the display device 300 and the auxiliary monitor 20 mounted on the ultrasonic probe 100.

In the meantime, as the image displayed on the auxiliary monitor 20, the entire image displayed on the display device 300 may be displayed and only a part of the image may be displayed.

In this case, as an embodiment, since the auxiliary monitor 20 is smaller than the display device 300, only a part of the image may be displayed, and for example, a central area of the image output on the display device 300 may be displayed. That is, the image is not displayed on the auxiliary monitor 20 by reducing the size of the image output on the display device 300, but only the central area thereof is displayed.

Accordingly, according to the present invention, since the operator using the ultrasonic waves may perform the procedure by examining the auxiliary monitor 20 put on the ultrasonic probe 100 while performing the ultrasonic procedure, an operation position and eyes of an operator coincide with each other so as for the operator to perform a convenient and accurate procedure. In particular, when an injection is given by using the ultrasonic probe, the present invention provides the effects in that the distance information up to the target point from the probe head 10 may be estimated by using the ultrasonic waves after finding a portion, to which the injection is given, while viewing the auxiliary monitor 20, and the advance or retreat degree of the piston of the syringe to be inserted may be controlled by using the estimated distance information.

The above-described embodiments of the present invention may be created by a computer executable program and implemented in a general use digital computer which operates the program using a computer readable recording medium. The computer readable recording medium includes a storing medium such as a magnetic storage medium (for example, a ROM, a floppy disk, and a hard disk), and an optical reading medium (for example, CD-ROM, a DVD).

The embodiments described above are examples and those skilled in the art can make various modifications and changes within the scope without departing from an essential characteristic of the present invention. Accordingly, the embodiments disclosed herein are intended not to limit but to describe the technical spirit of the present invention, and the scope of the spirit of the present invention is not limited to the embodiments. The protection scope of the present invention should be interpreted by the appended claims and all the technical spirits in the equivalent range thereto are intended to be embraced by the claims of the present invention.

1. An ultrasonic diagnostic device, comprising:
an ultrasonic probe which scans ultrasonic waves, converts the returned ultrasonic waves into an electric signal, and outputs a part or the entirety of an image corresponding to the converted electric signal to an auxiliary monitor mounted at one side thereof;
an ultrasonic diagnostic device body which receives the converted electric signal; and
a display device which is provided with the image corresponding to the electric signal from the ultrasonic diagnostic device body and outputs the entirety of the provided image,
wherein the ultrasonic probe includes an injection device which sucks or injects blood or an injection liquid at a predetermined target point within the image output to the auxiliary monitor.

2. The ultrasonic diagnostic device of claim 1, wherein the ultrasonic probe outputs a central area having a predetermined size in the image output through the display device to the auxiliary monitor.

3. The ultrasonic diagnostic device of claim 1, wherein the ultrasonic probe controls the injection device to estimate distance information up to the predetermined target point in the image output to the auxiliary monitor and suck or inject the blood or the injection liquid into the target point by using the estimated distance information.

4. An ultrasonic probe, comprising:
a probe head which scans ultrasonic waves and converts the returned ultrasonic waves into an electric signal;
a cable which transfers the converted electric signal to an ultrasonic diagnostic device body; and
an injection device which inserts a syringe at a predetermined target point in an image corresponding to the electric signal, and sucks or injects blood or an injection liquid through the inserted syringe.

5. The ultrasonic probe of claim 4, wherein an auxiliary monitor outputs a central area having a predetermined size in an image output through a display device mounted at one side of the probe head and connected to the ultrasonic diagnostic device body.

6. The ultrasonic probe of claim 4, wherein the auxiliary monitor is physically coupled with the probe head or physically separated from the probe head to be removably coupled thereto.

7. An ultrasonic probe having an auxiliary monitor mounted thereon, comprising:
a probe head which scans ultrasonic waves, and converts the returned ultrasonic waves into an electric signal;
an auxiliary monitor which outputs a part or the entirety of an image corresponding to the converted electric signal;
a cable which transfers the converted electric signal to an ultrasonic diagnostic device body, and receives an image corresponding to the electric signal transferred from the ultrasonic diagnostic device body; and
an injection device which inserts a syringe into a predetermined target point in the image output to the auxiliary monitor, and sucks or injects blood or an injection liquid through the inserted syringe.

8. The ultrasonic probe of claim 7, wherein the auxiliary monitor outputs a central area having a predetermined size in an image output through a display device mounted at one side of the probe head and connected to the ultrasonic diagnostic device body.

9. The ultrasonic probe of claim 7, wherein the injection device includes fixing means which fixes the syringe for sucking or injecting the blood or the injection liquid.

10. The ultrasonic probe of claim 7, wherein the injection device includes:
fixing means which fixes the syringe for sucking or injecting the blood or the injection liquid;
rotating means which rotates the syringe so as to control an angle at which a needle of the syringe is inserted; and
driving means which advances or retreats a piston of the syringe inserted at the target point.

11. The ultrasonic probe of claim 10, wherein the driving means estimates distance information up to the target point from the probe head by using the ultrasonic wave, and controls an advance or retreat degree of the piston of the syringe, which is to be inserted, by using the estimated distance information.

12. The ultrasonic probe of claim 7, wherein the ultrasonic probe further includes control means which estimates distance information from the probe head to the target point by using the ultrasonic wave, and
the injection device includes driving means which controls the advance or retreat degree of the piston of the syringe, which is to be inserted at the target point, by using the distance information estimated by the control means.
